(19) United States
(12) Patent Application Publication
(10) Pub. No.: US 2012/0053652 A1
(43) Pub. Date: Mar. 1, 2012

(54) METHOD AND SYSTEM FOR SENSING EXTERNAL MAGNETIC FIELDS USING A MULTI-FUNCTION COIL OF AN IMPLANTABLE MEDICAL DEVICE

(75) Inventors: Ali Dianaty, Northridge, CA (US); Ramez Shehada, La Mirada, CA (US)

(73) Assignee: PACESETTER, INC., Sylmar, CA (US)

(21) Appl. No.: 12/873,511

(22) Filed: Sep. 1, 2010

(51) Int. Cl. A61N 1/08

(52) U.S. Cl. 607/30

(57) ABSTRACT

An implantable medical device includes a lead, a monitoring module, a multi-function conductive (MFC) coil, and a field detection module. The monitoring module identifies cardiac events based on the cardiac signals and directs stimulus pulses to be delivered to the heart through one or more electrodes connected to the lead. The MFC coil has an electric characteristic that varies based on exposure of the coil to an external magnetic field. The field detection module detects exposure of the coil to the external magnetic field by applying a field detection signal to the coil and identifying a change in the electric characteristic of the MFC coil. The field detection module switches operation of the monitoring module to an MR safe mode based on the change that is identified.

### FIELD OF THE INVENTION

The present invention generally pertains to implantable medical devices and more particularly to methods and systems that switch modes of operation of an implantable medical device based on the presence of an external magnetic field.

### BACKGROUND OF THE INVENTION

An implantable medical device (IMD) is implanted in a patient to monitor, among other things, electrical activity of a heart and to deliver appropriate electrical therapy, as required. Implantable medical devices include pacemakers, cardioverters, defibrillators, implantable cardioverter defibrillators (ICD), and the like. The electrical therapy produced by an IMD may include pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm.

Strong magnetic fields may be produced by magnetic resonance (MR) imaging systems. For example, some known commercial MR imaging systems create magnetic fields on the order of 0.5 to 3.0 Tesla. When IMDs are exposed to external magnetic fields such as those of MR imaging systems, the fields may interfere with operation of the IMD. For example, an external magnetic field may generate magnetic forces on the IMD and on leads and electrodes of the IMD. These forces may induce electric charges or potential on the leads and electrodes. The electric charges can cause over- or under-sensing of cardiac signals in the electrodes and leads. For example, the charges may cause the electrodes and leads to convey signals to the IMD that are not cardiac signals but are treated by the IMD as cardiac signals. In another example, the charges may induce sufficient noise in the cardiac signals such that cardiac signals that are representative of a cardiac event go undetected by the IMD.

MR Imaging systems may generate external magnetic fields of different strengths, such as 0.5 Tesla, 0.7 Tesla, 1.0 Tesla, 1.2 Tesla, 1.5 Tesla, 3 Tesla, etc. Some IMDs may operate safely, while in certain modes, when exposed to lower strength magnetic fields. However, when IMDs are exposed to higher magnetic fields, the IMDs may be unable to reliably operate in a physiologic preferred manner in certain modes. In order to safely operate in some external magnetic fields, the IMDs may switch modes to an “MR safe mode” or a “magnet mode.”

In order to sense and detect external magnetic fields, some IMDs include additional magnetic sensors that are placed inside the IMD. These extra sensors that are added to the IMDs consume the limited volume inside the IMD housing. For example, some IMDs include Giant Magnetoresistance (GMR) sensors that are added to the interior of the IMDs to sense exposure of the IMDs to relatively weak external magnetic fields. The GMR sensors consume space in the IMD that may be used for other components. Additionally, the GMR sensors may be limited in that the sensors may be capable of only detecting exposure of the IMDs to relatively weak external magnetic fields. The sensors may be unable to differentiate between different external magnetic fields. For example, the sensors may be unable to distinguish between an external magnetic field generated by a relatively weak magnet and an external magnetic field generated by an MR imaging system.

A need exists for an IMD that includes a sensor capable of measuring exposure of the IMD to a variety of external magnetic fields while not consuming considerable space in the IMD.

### BRIEF SUMMARY OF THE INVENTION

In one embodiment, an implantable medical device (IMD) includes a lead, a monitoring module, a multi-function conductive (MFC) coil, and a field detection module. The monitoring module identifies cardiac events based on the cardiac signals and directs stimulus pulses to be delivered to the heart through one or more electrodes connected to the lead. The MFC coil has an electric characteristic that varies based on exposure of the coil to an external magnetic field. The field detection module detects exposure of the coil to the external magnetic field by applying a field detection signal to the coil and identifying a change in the electric characteristic of the MFC coil. The field detection module switches operation of the monitoring module to an MR safe mode based on the change that is identified

In accordance with another embodiment, a method for switching modes of an implantable medical device based on an external magnetic field includes sensing cardiac signals originating from a heart over electrodes positioned within the heart and monitoring the cardiac signals to identify cardiac events. The method also includes determining a change in an electric characteristic of a multi-function conductive coil disposed within the device, identifying exposure of the device to the external magnetic field based on the change in the electric characteristic of the coil, and switching operation of the device to an MR safe mode based on the change in the electric characteristic.

In accordance with another embodiment, a computer readable storage medium for use in an implantable medical device (IMD) includes instructions to direct a controller of the IMD to monitor cardiac signals of the heart to identify cardiac events, measure a change in an electric characteristic of a multi-function coil of the IMD, identify exposure of the device to an external magnetic field based on the change in the electric characteristic of the coil, and switch operation of the device to an MR safe mode based on the change in the electric characteristic.

### BRIEF DESCRIPTION OF THE DRAWINGS

The drawings illustrate generally, by way of example, but not by way of limitation, various embodiments discussed in the present document.

FIG. 1 illustrates an IMD coupled to a heart and including a multi-function conductive (MFC) coil.

FIG. 2 is an illustration of a telemetry circuit.

FIG. 3 is an illustration of field detection signals of the IMD shown in FIG. 1.

FIG. 4 illustrates changes in the field detection signals shown in FIG. 3 with respect to time.

FIG. 5 is a flowchart of a method for switching modes of the IMD 100 shown in FIG. 1 based on exposure to an external magnetic field.

FIG. 6 is a block diagram of exemplary internal components of the IMD shown in FIG. 1.

FIG. 7 illustrates a block diagram of example manners in which embodiments of the present invention may be stored, distributed, and installed on a computer-readable medium.

FIG. 8 is a first waveform template corresponding to a first level of magnetic field exposure.

FIG. 9 is a second waveform template corresponding to a second level of magnetic field exposure.

FIG. 10 is a waveform based on a field detection signal obtained from an MFC coil.

### DETAILED DESCRIPTION OF THE INVENTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated.

In accordance with certain embodiments, methods and systems are provided for automatically detecting entry of a patient who has an implantable medical device (IMD) into an external magnetic field, such as a field generated by a magnetic resonance imaging system, and automatically switching operation of the IMD to an MR safe mode. The IMD senses the external magnetic field using a multi-function conductive coil disposed in the IMD. For example, the IMD may identify exposure of the IMD to an external magnetic field using a coil in the IMD that is part of a telemetry circuit or a circuit that delivers stimulus pulses to the heart.

The IMD may be capable of distinguishing between external magnetic fields of different strengths and/or the proximity of the IMD to the external magnetic fields to determine if and when the IMD switches to a magnetic resonance (MR) safe mode. Switching operation of the IMD to a safe mode of operation when the IMD is in the magnetic field may prevent the IMD from malfunctioning. Once the patient and the IMD exit the magnetic field, the IMD may switch back to a normal mode of operation. For example, the IMD may return to the operating state used by the IMD prior to entering the MRI room.

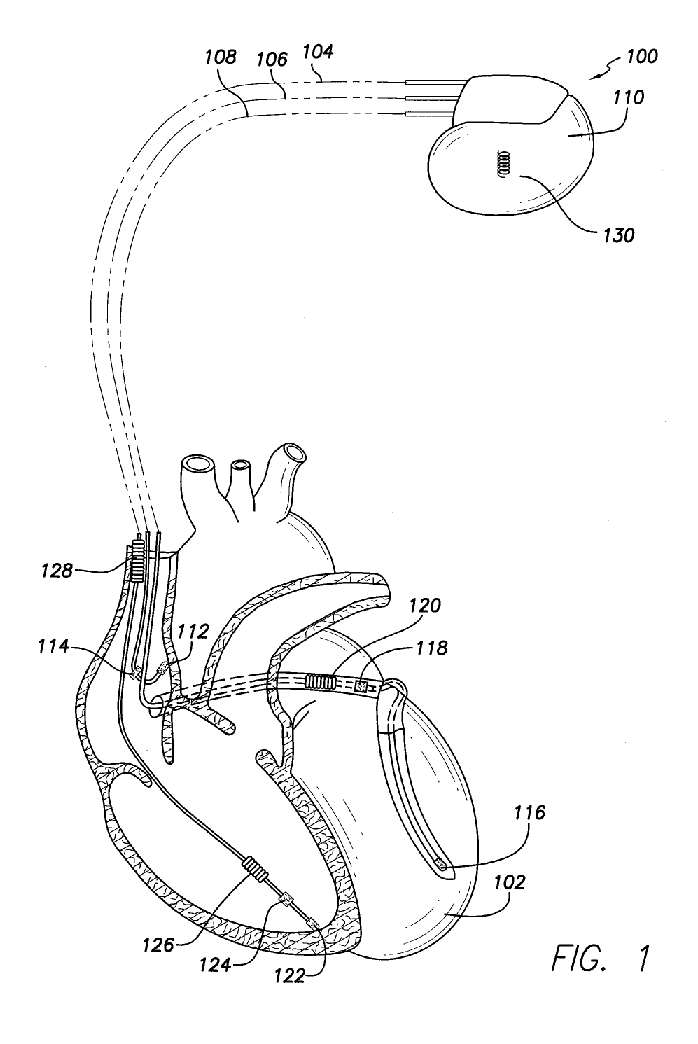

FIG. 1 illustrates an IMD 100 coupled to a heart 102 in accordance with one embodiment. The IMD 100 may be a cardiac pacemaker, an ICD, a defibrillator, an ICD coupled with a pacemaker, a cardiac resynchronization therapy (CRT) pacemaker, a cardiac resynchronization therapy defibrillator (CRT-D), and the like. The IMD 100 includes a housing 110 that is joined to several leads 104, 106, 108. The leads 104, 106, 108 are located at various locations of the heart 102, such as an atrium, a ventricle, or both, to measure cardiac signals of the heart 102. The leads 104, 106, 108 include the right ventricular (RV) lead 104, the right atrial (RA) lead 106, and the coronary sinus lead 108. Several electrodes 112, 114, 116, 118, 120, 122, 124, 126, 128 are coupled with the leads 104, 106, 108 for sensing cardiac signals and/or for delivering stimulus or stimulation pulses to the heart 102. The housing 110 may be one of the electrodes and is often referred to as the “can”, “case”, or “case electrode.”

The RV lead 104 is coupled with an RV tip electrode 122, an RV ring electrode 124, and an RV coil electrode 126. The RV lead 104 may include a superior vena cava (SVC) coil electrode 128. The right atrial lead 106 includes an atrial tip electrode 112 and an atrial ring electrode 114. The coronary sinus lead 108 includes a left ventricular (LV) tip electrode 116, a left atrial (LA) ring electrode 118 and an LA coil electrode 120. Alternatively, the coronary sinus lead 108 may be a quadropole lead that includes several electrodes disposed within the left ventricle. Leads and electrodes other than those shown in FIG. 1 may be included in the IMD 100 and positioned in or proximate to the heart 102.

The IMD 100 monitors cardiac signals of the heart 102 to determine if and when to deliver stimulus pulses to one or more chambers of the heart 102. The IMD 100 may deliver pacing stimulus pulses to pace the heart 102 and maintain a desired heart rate and/or shocking stimulus pulses to treat an abnormal heart rate such as tachycardia or bradycardia. The presence of a significantly strong external magnetic field such as that of MR imaging system may cause magnetic forces to interfere with the operation of the IMD 100.

The IMD 100 may switch modes of operation from a normal mode to a magnetic resonance (MR) safe mode when the IMD 100 enters the magnetic field. While in the MR safe mode, the IMD 100 may change the algorithms, software, or logical steps by which the cardiac signals are monitored and/or stimulus pulses are applied to the heart 102. For example, the IMD 100 may change which algorithms are used to identify an arrhythmia. Alternatively, the IMD 100 may cease measuring or sensing the cardiac signals. In another embodiment, the IMD 100 may ignore sensed cardiac signals and operate asynchronously when the IMD 100 is in the MR safe mode. Once the IMD 100 leaves the magnetic field, the IMD 100 may switch back to the normal mode of operation before again entering the magnetic field. In the normal mode, the IMD 100 may resume monitoring the cardiac signals as the IMD 100 did before the IMD 100 entered the magnetic field.

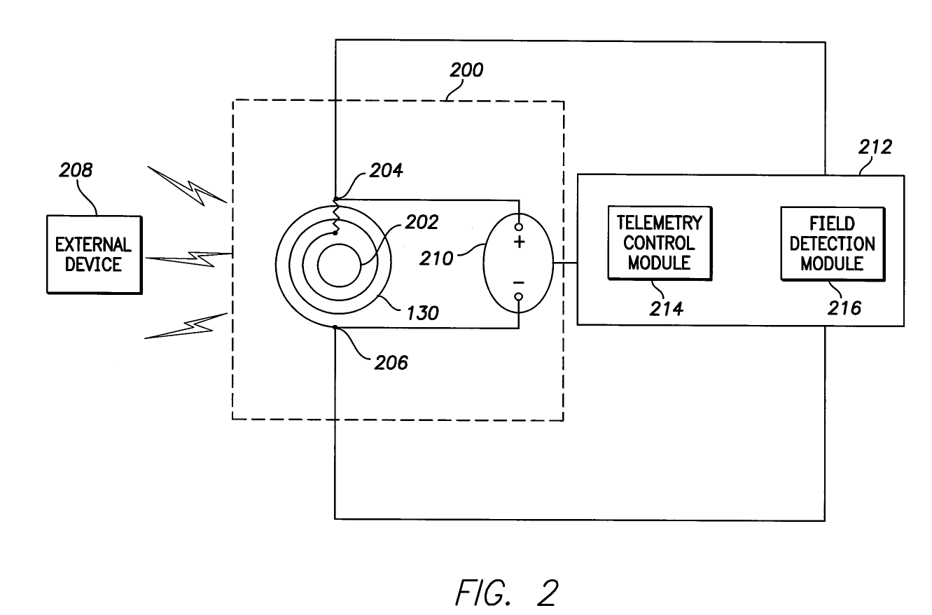

The IMD 100 includes a multi-function conductive (MFC) coil 130. The MFC coil 130 is capable of performing two or more functions of the IMD 100. For example, as one function, the MFC coil 130 may be a telemetry coil that is used to wirelessly communicate data with an external device 208 (shown in FIG. 2), such as an external programmer. Alternatively, the MFC coil 130 may be a transformer coil that steps up or increases an electric potential supplied by a power source 210 (shown in FIG. 2), such as a battery, of the IMD 100 prior to delivering the electric potential to the heart 102 as a shocking pulse via one or more of the electrodes 112, 114, 116, 118, 120, 122, 124, 126, 128. In another example, the MFC coil 130 may be an auxiliary coil that steps up or increases electric potential from the power source 210 prior to delivering the electric potential to the heart 102 as a stimulus or pacing pulse via one or more of the electrodes 112, 114, 116, 118, 120, 122, 124, 126, 128.

Another function of the MFC coil 130 is the use of the MFC coil 130 to determine when the IMD 100 is exposed to an external magnetic field. For example, an electric impedance characteristic of the MFC coil 130 may be used by the IMD 100 to determine the exposure of the MFC coil 130 to an external magnetic field, to determine the proximity of the MFC coil 130 to the external magnetic field, and/or to distinguish between external magnetic fields of different strengths. The use of a multi-function coil 130 to both sense exposure of the IMD 100 to an external magnetic field while also performing an additional function or purpose of the IMD 100 may avoid the need to include additional components in the IMD 100 to sense exposure to the external magnetic field.

FIG. 2 is an illustration of a telemetry circuit 200 in accordance with one embodiment. The telemetry circuit 200 is included in the IMD 100 (shown in FIG. 1) and may be used to wirelessly transmit data to and/or receive data from the external device 208. The telemetry circuit 200 includes the multi-function coil 130 helically wrapped around a core 202. The core 202 may be a cylindrical body of a ferrous material such as iron (Fe) or an iron alloy. Alternatively, the telemetry circuit 200 may not include the core 202.

In another embodiment, the MFC coil 130 is part of a circuit other than the telemetry circuit 200. For example, the MFC coil 130 may be a conductive coil that is included in a transformer or other circuit that increases an electric potential supplied by a power source, such as a battery, prior to supplying the electric potential to the heart 102 (shown in FIG. 1) as a stimulus pulse. Alternatively, the MFC coil 130 may be another conductive component of the IMD 100 (shown in FIG. 1) that has an electric impedance characteristic that changes based on exposure of the component to an external magnetic field.

The MFC coil 130 extends between a first end 204 and a second end 206. The power source 210 applies an electric potential or current to the MFC coil 130 across the first and second ends 204, 206 in order to drive the MFC coil 130 to transmit data to the external device 208. A programmable controller 212 of the IMD 100 (shown in FIG. 1) includes a telemetry control module 214 and a field detection module 216. The telemetry control module 214 communicates with the power source 210 and directs the power source 210 to apply the potential to the MFC coil 130 in order to control the data that is transmitted to the external device 208. The field detection module 216 may determine changes in an electric characteristic of the MFC coil 130. The changes in the electric characteristic may represent changes in the electric impedance characteristic of the MFC coil 130. For example, changes in an electric impedance characteristic of the MFC coil 130 or a voltage difference between the first and second ends 204, 206 of the MFC coil 130 may be used to distinguish between different external magnetic field strengths to which the MFC coil 130 is exposed. For example, the field detection module 216 may distinguish between external magnetic fields of less than 0.3 Tesla and fields that are at least 0.3 Tesla.

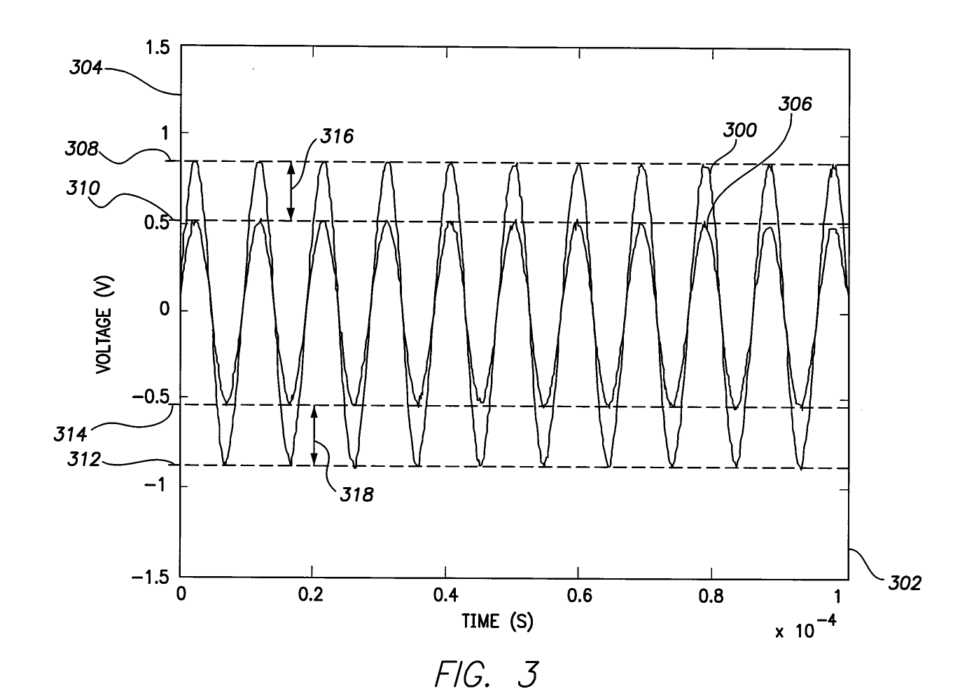

In accordance with one embodiment, the field detection module 216 may direct the power source 210 to apply a field detection signal 300 (shown in FIG. 3) to the MFC coil 130. This field detection signal 300 may be used to determine if the MFC coil 130 is exposed to an external magnetic field, to distinguish between different external magnetic fields, and/or to determine the proximity of the MFC coil 130 to the source of the external magnetic field.

The field detection signal 300 (shown in FIG. 3) that is applied to the MFC coil 130 may be an alternating signal, such as an alternating potential or current that periodically varies with respect to time. The field detection signal 300 may be applied as an alternating current across the first end 204 and the second end 206 of the MFC coil 130. The field detection module 216 may periodically examine or sample the field detection signal 300 across the first and second ends 204, 206 to determine if the field detection signal 300 has changed.

FIG. 3 is an illustration of field detection signals 300, 306 that are sensed across the first and second ends 204, 206 (shown in FIG. 2) of the MFC coil 130 (shown in FIG. 1). The field detection signal 300 represents an electric characteristic of the MFC coil 130 that is based on a current applied to the MFC coil 130. For example, the field detection signal 300 may represent voltages that are measured across or between the first and second ends 204, 206 when an alternating current is applied across the MFC coil 130 and when the MFC coil 130 is not exposed to an external magnetic field. The field detection signal 306 represents the electric characteristic of the MFC coil 130 that is measured when the MFC coil 130 is exposed to an external magnetic field. For example, the field detection signal 306 may represent voltages that are measured between or across the first and second ends 204, 206 of the MFC coil 130. Alternatively, the field detection signals 300, 306 may represent another electric characteristic, such as an impedance, resistance, and the like, of the MFC coil 130. For ease of reference, the field detection signal 300 may be referred to as an unexposed field detection signal 300 while the field detection signal 306 may be referred to as an exposed field detection signal 306.

The field detection signals 300, 306 are shown alongside a horizontal axis 302 representative of time and a vertical axis 304 representative of electric potential, or voltage. The field detection signals 300, 306 may be measured by applying an alternating current, such as a sinusoidal current, to the MFC coil 130 (shown in FIG. 1) and measuring the voltage across the first and second ends 204, 206 (shown in FIG. 2). For example, an alternating current may be applied to the first and second ends 204, 206 and the voltage difference between the first and second ends 204, 206 may be measured. Alternatively, the voltage difference may be obtained between two different locations along the MFC coil 130. For example, the alternating current may be applied at the first and second ends 204, 206 and the field detection signals 300, 306 may be measured as the voltage differences measured between two points along the MFC coil 130 that are located between the first and second ends 204, 206.

The alternating current that is applied to the MFC coil 130 (shown in FIG. 1) may have a waveform that is similar to the unexposed field detection signal 300. For example, as shown in FIG. 3, the current that is applied to the MFC coil 130 may be an alternating signal having a sinusoidal waveform with a frequency of approximately 100 kHz. Alternatively, the current may have a different waveform and/or frequency. For example, the current may have square, triangular, or saw-tooth waves in the waveform of the current. In another embodiment, the current may be a non-alternating current, such as a direct current.

An electric impedance characteristic of the MFC coil 130 (shown in FIG. 1) between the first and second ends 204, 206 (shown in FIG. 2) may vary or change based on exposure of the MFC coil 130 to an external magnetic field. For example, exposure of the MFC coil 130 to a relatively strong external magnetic field, such as a magnetic field generated by a magnetic resonance imaging (MRI) system, may saturate the MFC coil 130 and/or the core 202 (shown in FIG. 2). Saturation of the MFC coil 130 and/or core 202 may decrease an electric impedance characteristic of the MFC coil 130. The decrease or change in the electric impedance characteristic of the MFC coil 130 may depend on the strength of the external magnetic field and the proximity of the MFC coil 130 to the source of the external magnetic field. By way of example only, a stronger external magnetic field may decrease the electric impedance characteristic of the MFC coil 130 between the first and second ends 204, 206 more than a weaker external magnetic field. In another example, the electric impedance characteristic of the MFC coil 130 may be decreased when the MFC coil 130 moves closer to the source of the external magnetic field and increased when the MFC coil 130 moves away from the source of the external magnetic field.

The exposed field detection signal 306 is sensed by the field detection module 216 (shown in FIG. 2) at or across the first and second ends 204, 206 (shown in FIG. 2) of the MFC coil 130 (shown in FIG. 1). The field detection module 216 compares the unexposed and exposed field detection signals 300, 302 to identify one or more differences between the unexposed and exposed field detection signals 300, 302. Differences in the unexposed and exposed field detection signals 300, 302 may be based on a change in the unexposed field detection signal 300 that is caused by exposure of the MFC coil 130 to an external magnetic field, the strength of the external magnetic field, and/or the proximity of the MFC coil 130 to a source of the external magnetic field.

In the illustrated example, the MFC coil 130 (shown in FIG. 1) is saturated by exposure of the MFC coil 130 to a relatively strong external magnetic field, such as a magnetic field of at least 1.5 Tesla. Saturation of the MFC coil 130 may cause the electric impedance characteristic of the MFC coil 130 and the voltage measured between the first and second ends 204, 206 (shown in FIG. 2) to decrease. The exposed field detection signal 306 illustrates the drop or decrease in the voltage measured across the first and second ends 204, 206 when the MFC coil 130 is exposed to the external magnetic field. In one embodiment, the field detection module 216 (shown in FIG. 2) identifies a difference in the unexposed and exposed field detection signals 300, 302 as the decrease in the voltages from the unexposed field detection signal 300 to the exposed field detection signal 306.

The field detection module 216 may periodically sample the unexposed and exposed field detection signals 300, 306 in order to identify the upper or peak voltages of the unexposed and exposed field detection signals 300, 306. The upper or peak voltage of the unexposed field detection signal 300 may be the largest electric potential that is sampled between the first and second ends 204, 206 (shown in FIG. 2) of the MFC coil 130 (shown in FIG. 1) over a predetermined time window. The peak or upper voltage of the exposed field detection signal 306 may be the largest electric potential sampled between the first and second ends 204, 206 of the MFC coil 130 over the predetermined time window. As shown in FIG. 3, the peak voltages of the unexposed and exposed field detection signals 300 may be represented by amplitudes 308, 310. Alternatively, the amplitudes 308, 310 may correspond to different voltages of the unexposed and exposed field detection signals 300, 306. For example, the amplitudes 308, 310 may correspond to potentials other than the peak voltages of the unexposed and exposed field detection signals 300, 306.

Amplitudes 312, 314 may correspond to the valley voltages of the unexposed and exposed field detection signals 300, 306. For example, the amplitudes 312, 314 may be the smallest electric potential of the respective unexposed and exposed field detection signals 300, 306 that are sampled over a predetermined time window across or between the first and second ends 204, 206 (shown in FIG. 2) of the MFC coil 130 (shown in FIG. 1). Alternatively, the amplitudes 312, 314 may correspond to different voltages of the unexposed and exposed field detection signals 300, 306. For example, the amplitudes 312, 314 may correspond to potentials other than the valley voltages of the unexposed and exposed field detection signals 300, 306.

In one embodiment, the field detection module 216 (shown in FIG. 2) determines a difference 316, 318 between the amplitudes 308, 310 and/or between the amplitudes 312, 314. The difference 316 and/or 318 may be used by the field detection module 216 to determine if an electric impedance characteristic of the MFC coil 130 (shown in FIG. 1) has changed. In the example shown in FIG. 3, the amplitude 308 of the unexposed field detection signal 300 may decrease to the amplitude 310 of the exposed field detection signal 302 due to a change in the electric impedance characteristic of the MFC coil 130. The electric impedance characteristic of the MFC coil 130 may change because the MFC coil 130 has been exposed to an external magnetic field that, for example, can magnetically saturate the core 202 (shown in FIG. 2) of the MFC coil 130 and reduce effects of the external magnetic field on the inductance of the MFC coil 130.

The field detection module 216 (shown in FIG. 2) examines one or more parameters of the field detection signal 300 and/or 306 to identify the source and/or strength of an external magnetic field to which the MFC coil 130 (shown in FIG. 1) is exposed. For example, the field detection module 216 may be capable of measuring one or more parameters of the field detection signals 300, 306 to determine when the MFC coil 130 is exposed to relatively strong external magnetic fields, such as magnetic fields of at least 0.3 Tesla. Other examples of external magnetic fields that the field detection module 216 may be capable of detecting exposure of the MFC coil 130 to include fields of at least 0.3 Tesla, 0.5 Tesla, 1.5 Tesla, 3.0 Tesla, and the like.

In one embodiment, the field detection module 216 (shown in FIG. 2) determines an impedance change parameter based on the signals 300 and/or 306. The impedance change parameter may represent a change in an electrical impedance characteristic of the MFC coil 130 (shown in FIG. 1) due to exposure of the MFC coil 130 to an external magnetic field. The impedance change parameter may be based on the difference 316 between the amplitudes 308 and 310 and/or the difference 318 between the amplitudes 312 and 314. For example, the impedance change parameter may be a measurement of the difference 316, a measurement of the difference 318, an average or moving average of the difference 316 and/or 318, a median or moving median of the difference 316 and/or 318, a deviation of the difference 316 and/or 318, or some other statistical measure of the difference 316 and/or 318.

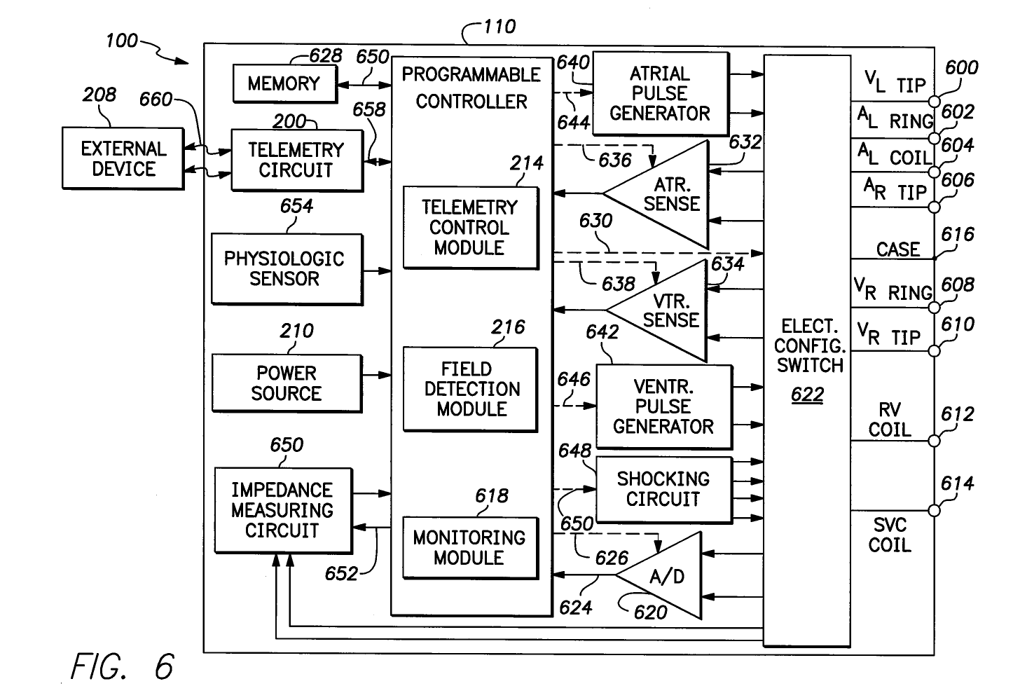

The impedance change parameter may be used by the field detection module 216 (shown in FIG. 2) to distinguish between different sources and/or strengths of external magnetic fields to which the MFC coil 130 (shown in FIG. 1) is exposed. For example, the field detection module 216 may use the impedance change parameter to distinguish between different magnets, different magnetic fields, and the like. In one embodiment, the field detection module 216 compares the impedance change parameter to one or more predetermined values of the impedance change parameter that are stored in a memory, such as a memory 628 (shown in FIG. 6) of the IMD 100 (shown in FIG. 1). The predetermined values of the impedance change parameter may represent absolute values of the impedance change parameter that are measured for different external magnetic field strengths. For example, a first value of the impedance change parameter may be measured for a MFC coil 130 that is proximate to a first source of an external magnetic field, such as a small hand-held magnet. A second value may be measured for a MFC coil 130 that is proximate to a second source, such as an activated MRI system. Additional values may be obtained. The impedance change parameter may be compared to these predetermined values in order to determine which of the predetermined values is closest to the impedance change parameter. In one embodiment, the source and/or strength of the external magnetic field that is associated with the predetermined value that is closest to the impedance change parameter is determined to be the source and/or strength of the external magnetic field for which the impedance change parameter is measured.

Alternatively, the impedance change parameter may be compared to one or more predetermined thresholds. The thresholds may be associated with different sources and/or strengths of external magnetic fields. If the impedance change parameter exceeds a first threshold but not a second threshold, then the impedance change parameter may indicate that the MFC coil 130 (shown in FIG. 1) is exposed to a source and/or strength of an external magnetic field that is associated with the first predetermined threshold. In another example, if the impedance change parameter exceeds both the first and second thresholds, then the impedance change parameter may indicate that the MFC coil 130 is exposed to a source and/or strength of an external magnetic field that differs from the source and/or strength associated with the first threshold.

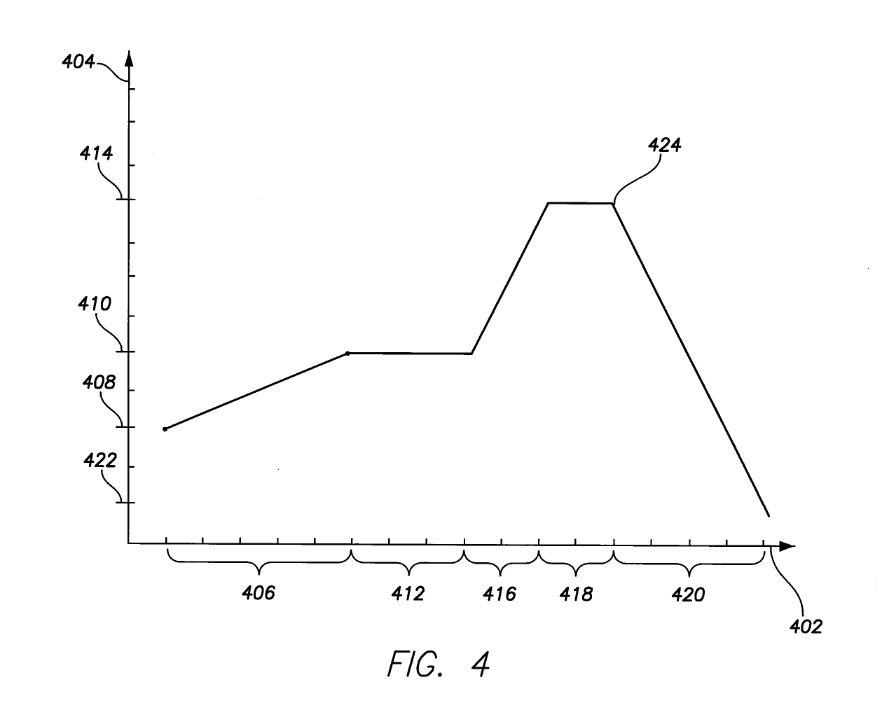

FIG. 4 illustrates a waveform 424 that is based on changes in the field detection signal 300 and/or 306 (shown in FIG. 3) with respect to time in accordance with one embodiment. The waveform 424 may represent the difference 316 (shown in FIG. 3), the impedance change parameter, or some other measure or calculation that is based on the field detection signal 300 and/or 306 (shown in FIG. 3). The waveform 424 is shown alongside a horizontal axis 402 representative of time and a vertical axis 404 representative of amplitude. In one embodiment, the vertical axis 404 may represent a voltage difference between the unexposed and exposed field detection signals 300, 306. The waveform 424 is illustrated as several interconnected linear portions. Alternatively, the waveform 424 may include one or more non-linear portions or sections.

As shown in FIG. 4, the waveform 424 can vary with respect to time. The waveform 424 may change with respect to time due to changes in the position of the patient and MFC coil 130 (shown in FIG. 1) relative to a source of an external magnetic field, changes in the strength of the external magnetic field, changes in the source of the external magnetic field, and the like. The waveform 424 may be used by the field detection module 216 (shown in FIG. 2) to distinguish between different sources and/or strengths of external magnetic fields.

The waveform 424 shown in FIG. 4 is composed of several segments 406, 412, 416, 418, 420. In the illustrated example, during an initial segment 406 of the waveform 424, the waveform 424 increases from a first value 408 to a second value 410. This increase in the waveform 424 may indicate that the patient and MFC coil 130 (shown in FIG. 1) are moving toward the source of an external magnetic field, such as an MRI system. Alternatively, this increase in the waveform 424 may indicate that a source of the external magnetic field, such as a small magnet, is moving toward the MFC coil 130, such as by being laid onto the patient's chest. In another example, the increase may indicate that the strength of the external magnetic field is increasing.

In contrast, during a subsequent segment 412 of the waveform 424, the waveform 424 remains approximately constant at the second value 410. The approximately constant waveform 424 may indicate that the patient, MFC coil 130 (shown in FIG. 1), and source of the external magnetic field are staying approximately stationary relative to each other and/or that the strength of the external magnetic field is remaining approximately constant.

The waveform 424 increases from the second value 410 to a third value 414 over a following segment 416 of the waveform 424. Similar to the increase in the waveform 424 during the initial segment 406, the increase in the waveform 424 during the following segment 416 may indicate that the MFC coil 130 (shown in FIG. 1) and the source of the external magnetic field are moving toward each other and/or that the strength of the external magnetic field is increasing. The waveform 424 remains approximately constant at the third value 414 during a subsequent segment 418. During a final segment 420, the waveform 424 decreases in value to a fourth value 422.

The field detection module 216 (shown in FIG. 2) may determine one or more morphology parameters of the waveform 424 to distinguish between the difference sources and/or strengths of external magnetic fields to which the MFC coil 130 (shown in FIG. 1) is exposed. One or more of the morphology parameters and the impedance change parameter may be examined to determine the source and/or strength of an external magnetic field.

In one embodiment, the morphology parameters identified by the field detection module 216 (shown in FIG. 2) includes a slope parameter. The slope parameter represents a rate of change or a slope of the waveform 424. For example, the slope parameter may be based on a change in the waveform 424 along the vertical axis 404 relative to a change in the waveform 424 along the horizontal axis 402 during a time window or time period. The field detection module 216 may calculate the slope parameter as a slope of the waveform 424 at one or more periodic sampling times, an average slope of the waveform 424, a moving average of the slope of the waveform 424, a median slope of the waveform 424 among several sampled slopes, a moving median slope of the waveform 424, and/or another statistical measure of the slopes of the waveform 424.

The slope parameter may indicate whether the patient and MFC coil 130 (shown in FIG. 1) are moving relative to a relatively large source of an external magnetic field, such as an MRI system, or a relatively small source, such as a hand-held magnet. The slope parameter may indicate whether the external magnetic field to which the MFC coil 130 is exposed is changing. In one embodiment, the slope parameter is compared to one or more predetermined thresholds to determine if the MFC coil 130 is moving toward a source of a relatively strong external magnetic field, such as an MRI system, or toward a source of a relatively weak external magnetic field, such as a small hand-held magnet. For example, the slope parameter may be compared to a predetermined threshold. If the slope parameter exceeds the threshold, then the slope parameter may indicate that the MFC coil 130 is moving relative to an external magnetic field or the source of the external magnetic field at a relatively fast rate. The slope parameter may exceed the threshold when the MFC coil 130 is moving too fast for a person to be moving toward an MRI system. For example, the relatively large slope parameter may indicate that the slope parameter is based on movement of a small magnet near a patient's chest. As a result, the field detection module 216 (shown in FIG. 2) may determine that the patient and/or source of the external magnetic field are moving too fast relative to each other for the patient to be walking toward a large source of the external magnetic field, such as an MRI system.

Alternatively, if the slope parameter does not exceed the threshold, then the slope parameter may indicate that the patient and MFC coil 130 (shown in FIG. 1) are moving more slowly toward the external magnetic field or source of the external magnetic field. For example, the slope parameter may indicate that the MFC coil 130 is moving toward an external magnetic field that is associated with the speed of a walking person. As a result, the field detection module 216 (shown in FIG. 2) may determine that the patient is walking toward a larger source of an external magnetic field, such as an MRI system.

In one embodiment, the field detection module 216 (shown in FIG. 2) compares the slope parameter to a plurality of thresholds that represent different speeds of relative movement between the MFC coil 130 (shown in FIG. 1) and the source of an external magnetic field. For example, a first threshold may represent a relatively slow speed associated with the walking of an elderly patient toward the source of the external magnetic field. A second threshold may represent a faster speed associated with the walking movement of a younger patient toward the source. A third threshold may represent an even faster speed associated with the relative movement between the patient and the source at a speed that is too fast for an active MRI system to be moved or for the patient to move toward the MRI system. The field detection module 216 may determine the source of the external magnetic field based on which of the thresholds are exceeded by the slope parameter.

The morphology parameters identified by the field detection module 216 (shown in FIG. 2) may include a polarity parameter. The polarity parameter represents the direction of change or slope of the waveform 424. The polarity parameter may be positive or negative based on whether the slope of the waveform 424 is positive or negative. By way of example only, the polarity parameter may have a value of 0 when the slope of the waveform 424 is negative and a value of 1 when the slope of the waveform 424 is positive. Alternatively, the polarity parameter may be 1 for negative slopes and 0 for positive slopes.

The polarity parameter may indicate whether the patient and MFC coil 130 (shown in FIG. 1) are moving toward or away from a source of an external magnetic field, such as an MRI system. For example, if the polarity parameter is positive, then the polarity parameter may indicate that the MFC coil 130 is moving toward a source of an external magnetic field. Alternatively, if the polarity parameter is negative, then the polarity parameter may indicate that the MFC coil 130 is moving away from the source.

The morphology parameters identified by the field detection module 216 (shown in FIG. 2) may include a shape parameter. One or more sections of the waveform 424 are compared to one or more waveform templates to determine the shape parameter. The shape parameter is a quantifiable degree or measurement to which the waveform 424 or section of the waveform 424 corresponds to or matches a waveform template. For example, the shape parameter may represent the correlation between the waveform 424 or a section of the waveform 424 and a waveform template. A larger shape parameter may represent a closer match or better correlation between the waveform 424 or section of the waveform 424 and a waveform template than a smaller shape parameter.

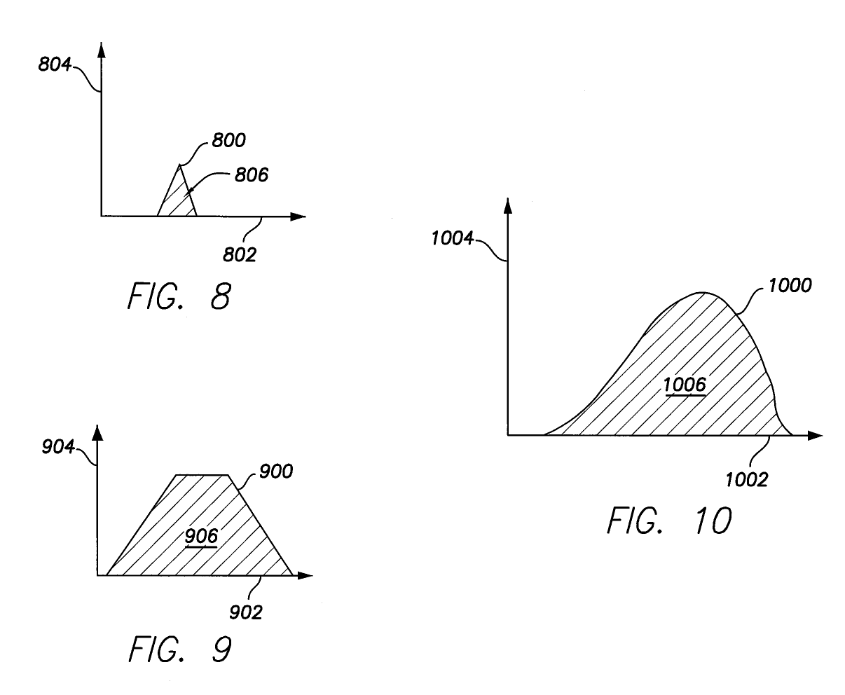

FIGS. 8 through 10 illustrate an example of how a waveform 1000 (shown in FIG. 10) may be compared to first and second predetermined waveform templates 800 (shown in FIG. 8), 900 (shown in FIG. 9) to determine a shape parameter for the waveform 1000. The waveform 1000 may be based on the field detection signal 300 and/or 306 (shown in FIG. 3), similar to the waveform 424 (shown in FIG. 4). The waveform 1000 and waveform templates 800, 900 are shown alongside horizontal axes 802, 902, 1002 representative of time and vertical axes 804, 904, 1004 representative of amplitude. The waveform templates 800, 900 are examples and one or more different waveform templates may be used. For example, the templates may be square waves, semi-circular waves, etc.

The waveform 1000 (shown in FIG. 10) is compared to the first and second waveform templates 800, 900 (shown in FIGS. 8 and 9) to calculate first and second shape parameters. In one embodiment, the first shape parameter may be calculated by comparing an area 806 (shown in FIG. 8) of the waveform template 800 (shown in FIG. 8) with an area 1006 (shown in FIG. 10) of the waveform 1000 (shown in FIG. 10). The area 806 includes the area bounded by the waveform template 800 and the horizontal axis 802 (shown in FIG. 8) and the area 1006 includes the area bounded by the waveform 1000 and the horizontal axis 1002 (shown in FIG. 10). The difference between the areas 806, 1006 may be a first shape parameter.

The second shape parameter may be calculated by comparing an area 906 (shown in FIG. 9) of the waveform template 900 (shown in FIG. 9) with the area 1006 (shown in FIG. 10) of the waveform 1000 (shown in FIG. 10). The area 906 includes the area bounded by the waveform template 900 and the horizontal axis 902 (shown in FIG. 9). The difference between the areas 906, 1006 may be a second shape parameter.

The first and second shape parameters may be compared to determine if the waveform 1000 (shown in FIG. 10) more clearly matches the waveform template 800 (shown in FIG. 8) or the waveform template 900 (shown in FIG. 9). For example, if the second shape parameter is smaller than the first shape parameter, then the waveform template 900 that is associated with the second shape parameter may be more closely correlated to the waveform 1000 than the waveform template 800 that is associated with the first shape parameter.

While the waveform 1000 (shown in FIG. 10) is compared to only two waveform templates 800, 900 (shown in FIGS. 8 and 9) in the above example, alternatively the waveform 1000 may be compared to more waveform templates. The different waveform templates 800, 900 may be associated with different sources and/or strengths of external magnetic fields. For example, the waveform template 800 may be associated with the MFC coil 130 (shown in FIG. 1) moving near or being in the proximity of a small magnet while the waveform template 900 may be associated with the MFC coil 130 moving near or being in the proximity of an activated MRI system. The field detection module 216 (shown in FIG. 2) determines the source and/or strength of the external magnetic field to which the MFC coil 130 is exposed based on which of the waveform templates 800, 900 are more closely correlated to the waveform 1000.

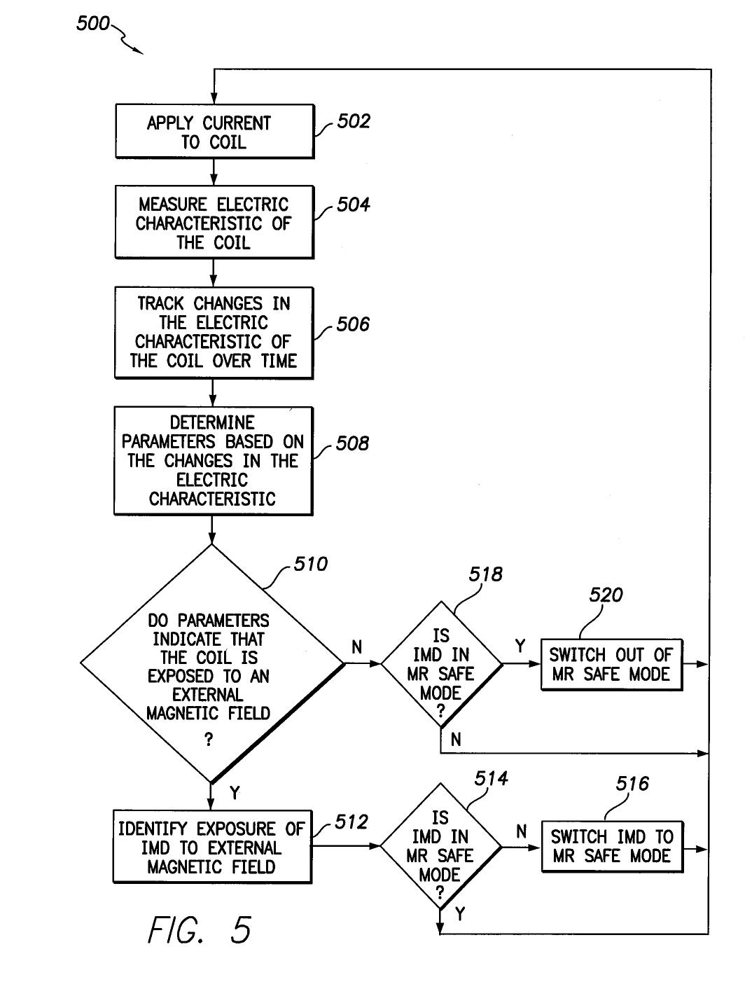

FIG. 5 is a flowchart of a method 500 for switching modes of an IMD 100 (shown in FIG. 1) based on exposure to an external magnetic field in accordance with one embodiment. At 502, an electric current is applied to a multi-function coil of the IMD 100. For example, the field detection signal 300 (shown in FIG. 3) may be applied to the MFC coil 130 (shown in FIG. 1).

At 504, an electric characteristic of the MFC coil 130 (shown in FIG. 1) is measured. The electric characteristic may be the field detection signal 306 (shown in FIG. 3) of the MFC coil 130.

At 506, changes in the electric characteristic of the MFC coil 130 (shown in FIG. 1) are tracked. For example, the differences 316 and/or 318 (shown in FIG. 3) between the field detection signals 300, 306 (shown in FIG. 3) may be measured over time. In one embodiment, the differences 316 and/or 318 are monitored to generate a waveform, such as the waveform 424 (shown in FIG. 4).

At 508, one or more parameters based on the changes in the electric characteristic of the MFC coil 130 (shown in FIG. 1) are determined. For example, an impedance change parameter and/or one or more morphology parameters may be determined, as described above.

At 510, a determination is made as to whether the parameters indicate that the MFC coil in the IMD is exposed to an external magnetic field. For example, the impedance change parameter and/or one or more of the morphology parameters may be compared to each other or to one or more thresholds to determine if the MFC coil 130 (shown in FIG. 1) is exposed to an external magnetic field. The parameters may be compared to each other and/or thresholds to identify the source and/or approximate strength of the external magnetic field, as described above.

If the parameters indicate that the MFC coil 130 (shown in FIG. 1) is exposed to an external magnetic field, such as an external magnetic field generated by an active MRI system, then flow of the method 500 may proceed to 512. On the other hand, if the parameters do not indicate that the MFC coil 130 is exposed to the external magnetic field, then flow of the method 500 continues to 518.

At 512, the IMD 100 (shown in FIG. 1) is identified as being exposed to an external magnetic field, such as the external magnetic field of an active MRI system. Alternatively, the IMD 100 may be identified as being exposed to an external magnetic field generated by a different source.

At 514, a determination is made as to whether the IMD 100 (shown in FIG. 1) is operating in an MR safe mode of operation. For example, a determination may be made to discern whether the IMD 100 previously was identified as being exposed to an external magnetic field generated by an MRI system and if the IMD 100 switched modes of operation to an MR safe mode of operation. If the IMD 100 already is in the MR safe mode of operation, then the IMD 100 may not need to switch to the MR safe mode. On the other hand, if the IMD 100 is not in the MR safe mode and is operating in a normal mode, for example, the IMD 100 may need to switch to the MR safe mode. If the IMD 100 already is in the MR safe mode of operation, flow of the method 500 returns to 502. The method 500 may continue in such a loop-wise manner until the IMD 100 is no longer exposed to the external magnetic field. On the other hand, if the IMD 100 is not in the MR safe mode of operation, flow of the method 500 continues to 516.

At 516, the IMD 100 (shown in FIG. 1) switches to the MR safe mode of operation. In the MR safe mode, the IMD 100 may change one or more of the algorithms, processes, methods, analyses, and the like, that are used to sense and monitor the cardiac signals of the heart 102 (shown in FIG. 1). For example, the IMD 100 may switch to a VOO mode when entering the MR safe mode. In the VOO mode, the IMD 100 stops sensing the cardiac signals of the heart 102 and paces the heart 102 at a predetermined rate. The IMD 100 may pace a ventricle at a fixed, predetermined lower rate interval without regard to the cardiac signals. The IMD 100 does not monitor or respond to any cardiac events that otherwise would be identified based on the cardiac signals. For example, the IMD 100 may ignore any cardiac signals that are indicative of a cardiac event when in the VOO mode and continue pacing the ventricle at the rate interval.

In another example, the IMD 100 (shown in FIG. 1) may switch to an AOO mode. In the AOO mode, the IMD 100 stops sensing the cardiac signals of the heart 102 (shown in FIG. 1) and paces one or both of the atria the heart 102 at a predetermined rate without regard to the cardiac signals. The IMD 100 does not monitor or respond to any cardiac events that otherwise would be identified based on the cardiac signals. Alternatively, the IMD 100 may switch to a DOO mode. In the DOO mode, the IMD 100 stops sensing and monitoring the cardiac signals. The IMD 100 paces both an atrium and a ventricle of the heart 102 at a fixed, predetermined rate. For example, the IMD 100 may apply stimulus pulses to an atrium at a first rate and, after a predetermined delay following each stimulus pulse to the atrium, apply a stimulus pulse to a ventricle. In another embodiment, the IMD 100 may continue to sense and monitor cardiac signals in the MR safe mode using one or more algorithms that differ from the algorithms used in the normal mode.

In another embodiment, the IMD 100 (shown in FIG. 1) may switch modes from the current mode of operation of the IMD 100 to a different mode based on one or more of the parameters determined at 508. One or more of the parameters may be compared to predetermined thresholds or waveform templates, as described above, that are associated with different external magnetic fields. The different external magnetic fields may be associated with different operating modes of the IMD 100. The different operating modes may, in turn, be associated with different algorithms, processes, methods, analyses, and the like, that are used to sense and monitor the cardiac signals of the heart 102 (shown in FIG. 1). Based on the parameters and the associated external magnetic fields, the IMD 100 may switch to a corresponding mode of operation and switch the methods used to sense and monitor signals of the heart 102.

Once the IMD 100 (shown in FIG. 1) switches to an MR safe mode of operation or to a mode of operation associated with an external magnetic field, flow of the method 500 continues back to 502. The method 500 proceeds back to 502 to continue monitoring changes in the electric characteristic of the MFC coil 130 (shown in FIG. 1) in order to determine if the external magnetic field to which the IMD 100 is exposed changes or if the IMD 100 exits the external magnetic field.

At 518, a determination is made as to whether the IMD 100 (shown in FIG. 1) is in an MR safe mode of operation. For example, the IMD 100 may already be operating in the MR safe mode when the parameters determined at 508 no longer indicate that the MFC coil 130 (shown in FIG. 1) is exposed to one or more external magnetic fields. If the IMD 100 is in the MR safe mode of operation or another mode of operation that is associated with an external magnetic field, then flow of the method 500 may proceed to 520.

At 520, the IMD 100 (shown in FIG. 1) switches out of the MR safe mode or the mode of operation that is associated with an external magnetic field. For example, the IMD 100 may switch to a normal mode of operation that was used by the IMD 100 prior to switching to the MR safe mode.

FIG. 6 is a block diagram of exemplary internal components of the IMD 100 in accordance with one embodiment. The IMD 100 includes the housing 110 that includes a left ventricle tip input terminal (V_{L} TIP) 600, a left atrial ring input terminal (A_{L} RING) 602, a left atrial coil input terminal (A_{L} COIL) 604, a right atrial tip input terminal (A_{R} TIP) 606, a right ventricular ring input terminal (V_{R} RING) 608, a right ventricular tip input terminal (V_{R} TIP) 610, an RV coil input terminal 612, and an SVC coil input terminal 614. A case input terminal 616 may be coupled with the housing 110. The input terminals 600 through 614 may be electrically coupled with the electrodes 112 through 128 (shown in FIG. 1).

The IMD 100 includes the programmable controller 212, which controls the operation of the IMD 100. The controller 212 (also referred to herein as a processor, processor module, or unit) typically includes a microprocessor, or equivalent control circuitry, and may be specifically designed for controlling the delivery of stimulation therapy and may further include RAM or ROM memory, logic and timing circuitry, state machine circuitry, and I/O circuitry. The controller 212 may include one or more modules and processors configured to perform one or more of the operations described above in connection with the method 500.

As described above, the controller 212 may include the telemetry control module 214 that controls application of electric potential or current to the telemetry circuit 200. The controller 212 includes the field detection module 216 that applies signals to the MFC coil 130 (shown in FIG. 1) of the telemetry circuit 200 and detects changes in the signals in order to determine exposure of the MFC coil 130 and IMD 100 to an external magnetic field. Alternatively, the field detection module 216 may apply the signals to another multi-function coil of the IMD 100, such as a transformer coil or an auxiliary coil that steps up or increases voltage supplied by the power source 210 prior to delivering the voltage to the heart 102 (shown in FIG. 1) as a stimulus pulse.

A monitoring module 618 of the controller 212 monitors cardiac signals of the heart 102 (shown in FIG. 1) to identify cardiac events. The monitoring module 618 employs one or more algorithms, processes, methods, and analyses to identify cardiac events such as cardiac waveforms, arrhythmias, and the like. The monitoring module 618 may measure intervals between cardiac events and/or calculate characteristics of cardiac waveforms to determine when particular cardiac events occur. The monitoring module 618 may automatically switch between different modes based on exposure of the IMD 100 to external magnetic fields. For example, the field detection module 216 may direct the monitoring module 618 to switch to or from an MR safe mode based on changes in the electric impedance characteristic of the MFC coil 130 (shown in FIG. 1).

The controller 212 receives signals from the electrodes 112 through 128 (shown in FIG. 1) via an analog-to-digital (ND) data acquisition system 620. The cardiac signals are sensed by the electrodes 112 through 128 and communicated to the data acquisition system 620. The cardiac signals are communicated through the input terminals 600 through 616 to an electronically configured switch bank, or switch, 622 before being received by the data acquisition system 620. The data acquisition system 620 converts the raw analog data of the signals obtained by the electrodes 112 through 128 into digital signals 624 and communicates the signals 624 to the controller 212. A control signal 626 from the controller 212 determines when the data acquisition system 620 acquires signals, stores the signals 624 in a memory 628, or transmits data to the external device 208 via the telemetry circuit 200.

The switch 622 includes switches for connecting the desired electrodes 112 through 128 (shown in FIG. 1) and input terminals 600 through 616 to the appropriate I/O circuits. The switch 622 closes and opens switches to provide electrically conductive paths between the circuitry of the IMD 100 and the input terminals 600 through 616 in response to a control signal 630. An atrial sensing circuit 632 and a ventricular sensing circuit 634 may be selectively coupled to the leads 104, 106, 108 (shown in FIG. 1) of the IMD 100 through the switch 622 for detecting the presence of cardiac activity in the chambers of the heart 102 (shown in FIG. 1). The sensing circuits 632, 634 may sense the cardiac signals that are analyzed by the controller 212. Control signals 636, 638 from the controller 212 direct output of the sensing circuits 632, 634 to the controller 212.

An atrial pulse generator 640 and a ventricular pulse generator 642 generate pacing stimulation pulses for delivery by the leads 104, 106, 108 (shown in FIG. 1) and the electrodes 112 through 128 (shown in FIG. 1). The atrial and ventricular pulse generators 640, 642 may include dedicated, independent pulse generators, multiplexed pulse generators, or shared pulse generators. The pulse generators 640, 642 may include one or more conductive coils that may be used in a manner similar to the MFC coil 130 (shown in FIG. 1) of the telemetry circuit 200. For example, the pulse generators 640, 642 may include a transformer or auxiliary coil over which a signal may be applied. The controller 212 may measure or identify changes in the signal between ends of the coil and use the changes to determine when the IMD 100 is exposed to an external magnetic field. The pulse generators 640, 642 are controlled by the controller 212 via appropriate control signals 644, 646 respectively, to trigger or inhibit the stimulation pulses.

In the case where the IMD 100 is intended to operate as an implantable cardioverter/defibrillator (ICD) device, the IMD 100 may detect the occurrence of an arrhythmia, and automatically apply an appropriate electrical shock therapy to the heart aimed at terminating the detected arrhythmia. To this end, the controller 212 further controls a shocking circuit 648 by way of a control signal 650. The shocking circuit 648 generates shocking pulses that are applied to the patient's heart 102 (shown in FIG. 1) through at least two shocking electrodes, such as the coil electrode 120 (shown in FIG. 1), the RV coil electrode 126 (shown in FIG. 1), and/or the SVC coil electrode 128 (shown in FIG. 1). The shocking circuit 648 may include a coil that is used to step up or increase an electric potential supplied to the shocking circuit 648 by the power source 210. The coil of the shocking circuit 648 also may be used to determine when the IMD 100 is exposed to an external magnetic field. As described above, the field detection module 216 of the controller 212 may apply an electric current to the coil of the shocking circuit 648 and measure or identify changes in electric characteristics of the coil as the current is conveyed through the coil. These changes in the electric characteristic may be used by the field detection module 216 to determine if the IMD 100 is exposed to an external magnetic field and/or to distinguish between different external magnetic fields.

An impedance measuring circuit 650 is enabled by the controller 212 via a control signal 652. The impedance measuring circuit 650 may be electrically coupled to the switch 622 so that an impedance vector between any desired pairs of electrodes 112 through 128 (shown in FIG. 1) may be obtained. The IMD 100 includes a physiologic sensor 654 that may be used to adjust pacing stimulation rate according to the exercise state of the patient.

The memory 628 may be embodied in a computer-readable storage medium such as a ROM, RAM, flash memory, or other type of memory. The controller 212 is coupled to the memory 628 by a suitable data/address bus 656. The memory 628 may store programmable operating parameters and thresholds used by the controller 212, as required, in order to customize the operation of IMD 100 to suit the needs of a particular patient. The operating parameters of the IMD 100 and thresholds may be non-invasively programmed into the memory 628 through the telemetry circuit 200 in communication with the external device 208, such as a trans-telephonic transceiver or a diagnostic system analyzer. The telemetry circuit 200 is activated by the controller 212 by a control signal 658. The telemetry circuit 200 allows intra-cardiac electrograms, cardiac waveforms of interest, thresholds, status information relating to the operation of IMD 100, and the like, to be sent to the external device 208 through an established communication link 660.

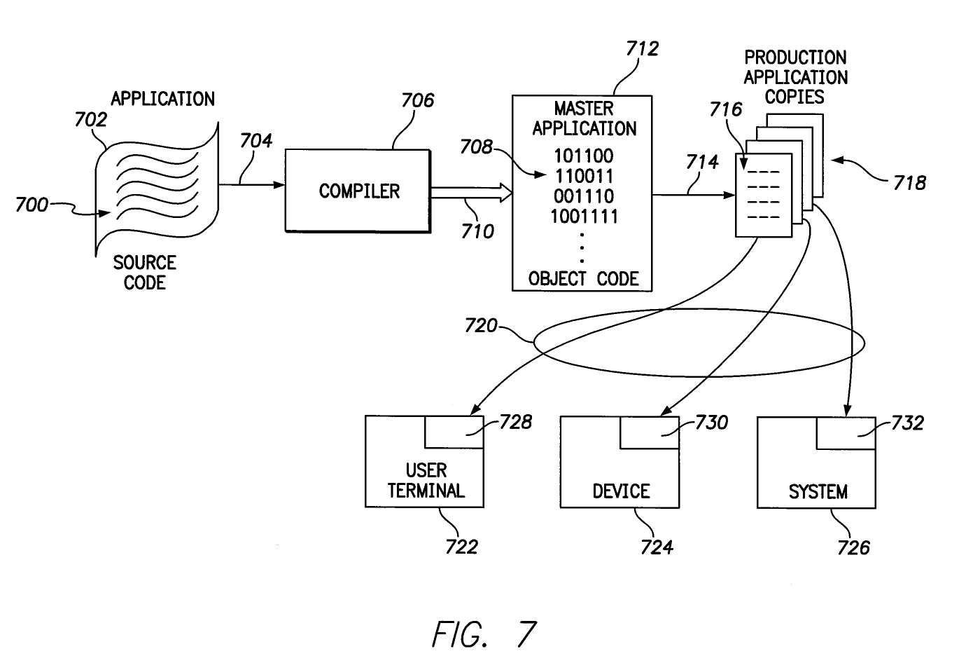

FIG. 7 illustrates a block diagram of example manners in which embodiments of the present invention may be stored, distributed, and installed on a computer-readable medium. In FIG. 7, the “application” represents one or more of the methods and process operations discussed above. The application is initially generated and stored as source code 700 on a source computer-readable medium 702. The source code 700 is then conveyed over path 704 and processed by a compiler 706 to produce object code 708. The object code 708 is conveyed over path 710 and saved as one or more application masters on a master computer-readable medium 712. The object code 708 is then copied numerous times, as denoted by path 714, to produce production application copies 716 that are saved on separate production computer-readable media 718. The production computer-readable media 718 are then conveyed, as denoted by path 720, to various systems, devices, terminals and the like.

A user terminal 722, a device 724, and a system 726 are shown as examples of hardware components, on which the production computer-readable media 718 are installed as applications (as denoted by 728, 730, 732). For example, the production computer-readable medium 718 may be installed on the IMD 100 (shown in FIG. 1) and/or the controller 212 (shown in FIG. 2). Examples of the source, master, and production computer-readable media 702, 712, and 718 may include tangible and non-transitory devices or media such as CDROM, RAM, ROM, Flash memory, RAID drives, memory on a computer system, and the like. Examples of the paths 704, 710, 714, 720 include, but are not limited to, network paths, the internet, Bluetooth, GSM, infrared wireless LANs, HIPERLAN, 3G, satellite, and the like.

The operations noted in FIG. 7 may be performed in a widely distributed manner world-wide with only a portion thereof being performed in the United States. For example, the application source code 700 may be written in the United States and saved on a source computer-readable medium 702 in the United States, but transported to another country (corresponding to path 704) before compiling, copying and installation. Alternatively, the application source code 700 may be written in or outside of the United States, compiled at a compiler 706 located in the United States and saved on a master computer-readable medium 712 in the United States, but the object code 708 transported to another country (corresponding to path 714) before copying and installation. Alternatively, the application source code 700 and object code 708 may be produced in or outside of the United States, but production application copies 716 produced in or conveyed to the United States (for example, as part of a staging operation) before the production application copies 716 are installed on user terminals 722, devices 724, and/or systems 726 located in or outside the United States as applications 728, 730, 732.

As used throughout the specification and claims, the phrases “computer-readable medium” and “instructions configured to” shall refer to any one or all of (i) the source computer-readable medium 702 and source code 700, (ii) the master computer-readable medium and object code 708, (iii) the production computer-readable medium 718 and production application copies 716 and/or (iv) the applications 728, 730, 732 saved in memory in the terminal 722, device 724, and system 726.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable medical device comprising:
a lead including electrodes configured to be positioned to sense cardiac signals of a heart;
a monitoring module configured to identify cardiac events based on the cardiac signals and to direct stimulus pulses to be delivered to the heart through one or more of the electrodes;
a multi-function conductive (MFC) coil having an electric characteristic that varies based on exposure of the MFC coil to an external magnetic field; and
a field detection module configured to: detect exposure of the MFC coil to the external magnetic field by applying a field detection signal to the MFC coil, identify a change in the electric characteristic of the MFC coil, and switch operation of the monitoring module to an MR safe mode based on the change that is identified.

2. The implantable medical device of claim 1, wherein the field detection module identifies the change in the electric characteristic based on an electric impedance characteristic of the MFC coil.

3. The implantable medical device of claim 1, wherein the MFC coil is at least one of a telemetry coil that is used to wirelessly communicate data with an external device or a transformer coil that increases an electric potential supplied by a power source prior to delivering the electric potential to the heart as one or more of the stimulus pulses.

4. The implantable medical device of claim 1, further comprising a telemetry circuit to wirelessly communicate data with an external device, the MFC coil forming part of the telemetry circuit to at least one of transmit or receive the data to and from the external device.

5. The implantable medical device of claim 1, further comprising a battery coupled to the MFC coil, the battery configured to supply an electric potential to the heart as one or more of the stimulus pulses, the MFC coil configured to increase the electric potential prior to being delivered to the heart as the one or more of the stimulus pulses.

6. The implantable medical device of claim 1, wherein the field detection module identifies the change in the electric characteristic of the MFC coil when the MFC coil is exposed to the external magnetic field of at least 0.3 Tesla.

7. The implantable medical device of claim 1, wherein the field detection module identifies exposure of the MFC coil to the external magnetic field based on one or more of an impedance change parameter or a morphology parameter, the impedance change parameter based on the electric characteristic and the morphology parameter based on a waveform that is derived from changes in the electric characteristic with respect to time.

8. The implantable medical device of claim 1, wherein the field detection module determines the change in the electric characteristic by applying an alternating current to the MFC coil and measuring a voltage difference between points of the MFC coil.

9. The implantable medical device of claim 8, wherein the MFC coil extends between opposite ends and the field detection module measures the voltage difference in the alternating current between the ends of the MFC coil.

10. The implantable medical device of claim 1, wherein the detection module stops monitoring the cardiac signals when switched to the MR safe mode.

11. A method for switching modes of an implantable medical device based on an external magnetic field, the method comprising:
sensing cardiac signals originating from a heart;
monitoring the cardiac signals to identify cardiac events;
determining a change in an electric characteristic of a multi-function conductive (MFC) coil disposed within the device;
identifying exposure of the device to the external magnetic field based on the change in the electric characteristic of the MFC coil; and
switching operation of the device to an MR safe mode based on the change that is identified.

12. The method of claim 11, wherein the MFC coil is a telemetry coil that wirelessly communicates data with an external device.

13. The method of claim 11, further comprising using the MFC coil to increase an electric potential of stimulus pulses and applying the stimulus pulses to the heart.

14. The method of claim 11, wherein identifying exposure comprises detecting a rate at which the electric characteristic of the MFC coil changes over a time period and identifying the exposure of the device to the external magnetic field based on the rate.

15. The method of claim 11, wherein identifying exposure comprises determining a morphology parameter of a waveform that is derived from a plurality of the changes in the electric characteristic over time.

16. The method of claim 11, wherein determining a change in an electric characteristic comprises applying an alternating current to the MFC coil and measuring a voltage difference between points of the MFC coil, the change in the electric characteristic based on the voltage difference.

17. The method of claim 16, wherein identifying exposure comprises measuring the voltage difference between opposite ends of the MFC coil.

18. The method of claim 11, wherein switching operation of the device comprises stopping the monitoring of the cardiac signals when the device is switched to the MR safe mode.

19. A computer readable storage medium for use in an implantable medical device having electrodes configured to sense cardiac signals of a heart, a programmable controller, and a multi-function conductive (MFC) coil, the computer readable storage medium comprising instructions to direct the controller to:
monitor cardiac signals of the heart to identify cardiac events;
measure a change in an electric characteristic of the MFC coil;
identify exposure of the device to an external magnetic field based on the change in the electric characteristic of the MFC coil; and
switch operation of the device to an MR safe mode based on the change that is measured.

20. The computer readable storage medium of claim 19, wherein the instructions direct the controller to at least one of wirelessly communicate data with an external device using the MFC coil or increase an electric potential of a stimulus pulse using the MFC coil prior to delivering the stimulus pulse to the heart.

21. The computer readable storage medium of claim 19, wherein the instructions direct the controller to detect a rate at which the electric characteristic of the MFC coil changes over a time period and identify the exposure of the device to the external magnetic field based on the rate.

22. The computer readable storage medium of claim 19, wherein the instructions direct the controller to apply a field detection signal to the MFC coil and measure a voltage difference between points of the MFC coil, the change in the electric characteristic based on the voltage difference.

23. The computer readable storage medium of claim 22, wherein the instructions direct the controller to measure the voltage difference between opposite ends of the MFC coil.

24. The computer readable storage medium of claim 19, wherein the instructions direct the controller to stop monitoring the cardiac signals when switched to the MR safe mode.

25. The computer readable storage medium of claim 19, wherein the instructions direct the controller to identify exposure of the device to the external magnetic field by determining at least one of an impedance change parameter of the MFC coil or one or more morphology parameters of a waveform comprising a plurality of the changes in the electric characteristic over time.
